(19) United States
(12) Patent Application Publication
(10) Pub. No.: US 2015/0257727 A1
(43) Pub. Date: Sep. 17, 2015

(54) DEVICE FOR THE CALIBRATION OF A QUANTITATIVE COMPUTED TOMOGRAPHY APPARATUS

(71) Applicant: BIOTECHNOLOGY INSTITUTE, I MAS D, S.L., Vitoria (Alava) (ES)

(72) Inventor: Eduardo ANITUA ALDECOA, Vitoria (Alava) (ES)

(21) Appl. No.: 14/645,723

(22) Filed: Mar. 12, 2015

(30) Foreign Application Priority Data
Mar. 13, 2014 (ES) P 201430341

(51) Int. Cl. A61B 6/00; A61B 6/03; A61B 6/14

(52) U.S. Cl. CPC A61B 6/583; A61B 6/145; A61B 6/035

(57) ABSTRACT

Device (10; 30; 50; 70) for calibration of a quantitative computed tomography apparatus, which includes a body (12; 32; 52; 72) and several known-density elements (13; 33; 53; 73) attached to the body and made of different materials and in different densities from each other and different from the body. The body (12; 32; 52; 72) is configured to be placed in the mouth or on another part of a person's head, with the known-density elements (13; 33; 53; 73) arranged in the region of the person's teeth. The device enables a quantitative computed tomography apparatus to adjust its calculations so as to convert the radiodensity units of the tomographic image into bone mineral density units, by knowing the exact densities of certain points of the image, corresponding to the points where the known-density elements (13; 33; 53; 73) are located.

### TECHNICAL FIELD

The invention relates to a device for the calibration of a quantitative computed tomography apparatus. The device is inserted into the mouth of a person and includes portions of materials of known densities.

### PRIOR ART

Computed tomography (CT) is an image-capturing technology that uses X-rays, in conjunction with the capacity of a computer processor, to obtain tomographic images of an object. Tomographic images are consecutive images of an object taken along an axial direction, by way of slices of the object, where the images have different levels of grey depending on the radiodensity of the object scanned. The most frequently used unit of measurement for radiodensity is the Hounsfield unit (HU). Tomographic images are currently processed by computers, which are capable of processing tomographic images in order to obtain the necessary information and to view them in the most suitable way for the field of the technique in question. In the medical field, for example, image reconstruction and processing software has evolved to currently enable the succession of flat images to be transformed into three-dimensional images in which some tissues are distinguished from others, and in which the tissues to be displayed are even selectable. Other improvements in computed tomography techniques are helical (or spiral) technology, which enables more accurate images to be obtained, and multislice technology, in which the number of sensors is increased, allowing multiple images to be obtained simultaneously and increasing the speed of obtaining volumetric imaging, which can even be obtained in real time. The ultimate goal is to obtain higher quality images in less time and requiring lower radiation for the patient.

In the field of dental medicine, computed tomography is currently used for many purposes, one of which is to acquire a perfect understanding of the bone anatomy of a patient so as to carry out optimal planning for placing one or more implants and prostheses. Sagittal slices generated by computed tomography enable greater precision to be achieved in placing the implant and in detecting the location of the lower (inferior) dental canal than conventional orthopantomography or panoramic radiography. This allows the risk of injury to the inferior alveolar (dental) nerve to be reduced and the risk of inserting the implant into structures such as the sublingual or submandibular fossas (foveas), which are not seen in conventional orthopantomography, also to be reduced.

To do this, a kind of computed tomography known as quantitative computed tomography, consisting of a medical technology that can measure the bone density of a bone or set of bones, is normally used. The scanner that performs the quantitative computed tomography has a calibration functionality that enables the radiodensity units of tomographic images (usually Hounsfield units) to be converted into bone mineral density values, thus allowing quantitative bone mineral density values to be obtained; calibration also allows the scale of greys of tomographic images to be normalized, making it possible for small changes in bone volume and density to be observed (as changes in the levels of grey in images). The quantitative computed tomography technique is being used very successfully, since it is able to distinguish different parts of the bone, such as cortical (compact) bone and trabecular (cancellous/spongy) bone, from each other. Distinguishing trabecular bone from cortical bone is of vital importance, since the metabolic activity of trabecular bone is 3 to 10 times higher than that of cortical bone and, therefore, trabecular bone is where greater variability of density changes will take place over time.

Calibration of tomographic imaging so as to convert radiodensity information into bone mineral density values is a key step for obtaining quality quantitative computed tomographies. Different methods and systems of carrying out calibration are known in prior art.

There are two traditional calibration techniques: non-simultaneous and simultaneous calibrations, depending on whether they are performed prior to placing the patient or with the patient in situ. Non-simultaneous calibrations are those that are performed as part of the periodic maintenance of the computed tomography apparatus, to avoid errors arising from technical defects in the apparatus itself. Simultaneous calibrations are performed by placing a calibration phantom that has parts with known densities next to the patient, such as epoxy resin parts of known density or cortical bone chips of known density, for instance; the apparatus takes images of the patient and adjusts bone mineral density calculations so that the areas of the image where the devices with known densities are located have quantitative density values matching the previously known densities of these devices. However, it has been proven that conventional simultaneous calibration techniques do not provide accurate calibration.

Several factors can make calibration of quantitative computed tomography apparatus necessary:
Object-dependent factors: the superimposition of soft tissue and other dispersion factors present in the mouth (denture/prosthesis, amalgams, etc.) cause contamination in the live image obtained and can only be overcome by adapting the calibration design.
Machine-dependent factors: it has been proven that the scale of HU units varies depending on the type of scanner used, due to the lack of uniformity of the X-ray beam. It is remedied by calibration of the scanner apparatus.
Factors arising from image digitization and compression: CT images are currently digitized. Current image compression systems, such as ZIP, JPEG and DICOM, which, despite being necessary for filing, data transmission and fast program operation, present a greater or lesser inherent loss of information that sometimes affects the greyscale on which images are based. This causes an alteration in the accuracy of measurements, especially in densitometry measurements, which are entirely dependent on the degree of grey.
Factors arising from the software used: there are currently many software programmes capable of measuring density. Comparison as regards density measurement in HU units by different programmes is difficult to achieve because of the different approaches that might occur, such as the inclusion of cortical bone in the ROI (Region Of Interest), the use of different image compression methods with data loss, the inclusion of reformatted images such as sagittal slices and the size of the ROI.
Factors arising from parameters: exposure time, kilovoltage and miliamperage. Changes or fluctuations in these parameters lead to inaccuracies in bone mass estimation.
Receiver-dependent factors: artifacts caused by items close to the study area, such as metal fillings, bridges with a metal content, etc. The importance of performing scanning with the mouth open and the jaws well separated, in order to avoid metal artifacts in one or other area, should be emphasized at this point. Even so, there are always alien materials and or even materials from the patient him/herself (like tooth enamel) that, due to their high X-ray absorption, partly artefact images, affecting the greyscale.
Operator-dependent factors: it is worth mentioning that a great variability can take place in dependence of the operator, i.e., the radiology technician performing the scanning, and how he or she is able to reduce the above factors.
Factors arising from patient positioning: poor patient positioning may lead to errors in bone density readings.

It is an object of the present invention to design a calibration phantom or device for quantitative computed tomography apparatus that is specially designed for applications in dental medicine, in order to facilitate carrying out in situ or non-simultaneous calibrations with the patient.

### BRIEF DESCRIPTION OF THE INVENTION

In order to achieve the objectives mentioned above, a device for calibration of a quantitative computed tomography apparatus is proposed, which comprises a body with two or more known-density elements attached to it. The known-density elements are made of different materials and have different densities from each other. Moreover, the known-density elements have different densities from the body itself, and are made of different materials from the material or materials of which the body is manufactured. The body, in turn, is configured to be at least partially placed inside the mouth or coupled to another part of a person's head, and so that the known-density elements are arranged in the region of the person's teeth. The device according to the invention can be coupled to a person's head, either outside or at least partially inserted inside the mouth, allowing a quantitative computed tomography of the head to be performed together with the device, so as to obtain an image of the patient's bones and of the known-density elements close to the teeth. The known-density elements have a previously known density, allowing the quantitative computed tomography apparatus control software to self-calibrate so that the quantitative tomographic images provide bone mineral density values, at the points where the known-density elements are located, equal to the previously known densities.

In certain embodiments the known-density elements are arranged inside the person's mouth, behind the teeth, whereas in other embodiments they are arranged outside the person's mouth, around the area of the teeth.

In preferred embodiments, the device is made of a combination of materials that enables optimal calibration to be obtained for subsequently measuring of the bone mineral density of a patient, and at the same time the device is fully sterilizable so that it may be used with different patients.

### BRIEF DESCRIPTION OF THE DRAWINGS

Details of the invention can be seen in the accompanying drawings, which do not seek to restrict the scope of the invention:

FIG. 1 shows a perspective view of a first embodiment of the invention.

FIG. 2 shows a perspective view of a second embodiment of the invention.

FIG. 3 shows a perspective view of a third embodiment of the invention.

FIG. 4 shows a perspective view of a fourth embodiment of the invention.

### DETAILED DESCRIPTION OF THE INVENTION

The invention relates to a device to be placed on a patient and enable calibration of a quantitative computed tomography apparatus, which is placed in order to perform a scan of the patient's mouth. The device according to the invention is prepared to be coupled to the patient's head or mouth and has various possible configurations, some of which are shown in the figures accompanying this description.

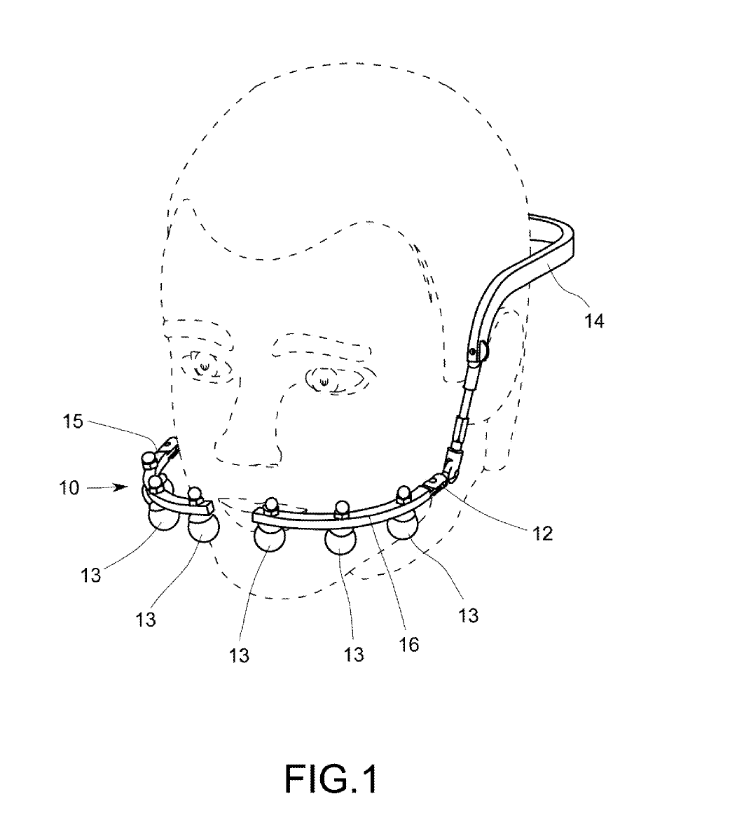

FIG. 1 shows a first embodiment of the invention, consisting of a device (10) for calibrating a quantitative computed tomography apparatus, where the said device (10) is shown, in the figure, placed on a patient's head. The device (10) includes a body (12) with six known-density elements (13) attached to it. In this case the known-density elements (13) are six spheres made of sterilizable plastic material and capable of being subjected to an X-ray scanner without deteriorating. The six known-density elements (13) are not all made of the same material and do not have the same density, although there can be some known-density elements (13) that have the same density and are made of the same material. In this embodiment, for example, the three known-density elements (13) on one side of the face may be made respectively of three materials and have different densities and, at the same time, the three known-density elements (13) arranged on the opposite side of the face might be made symmetrically. As shown in the figure, the body (12) is shaped to be attached externally to the patient's head, with the known-density elements (13) arranged in the region of this person's teeth. In the embodiment illustrated, the external attachment to the head is provided by a cranial engagement portion (14) included in the body (12), which is configured in size and shape to engage with and be supported on an area of the head of the corresponding person's skull. In the embodiment illustrated, for example, the cranial engagement portion (14) is configured in size and shape to be arranged above the ears and behind the head of the patient, while two front portions (15, 16) extend along the sides of the patient's face and support the known-density elements (13) so that they are placed externally along the patient's teeth.

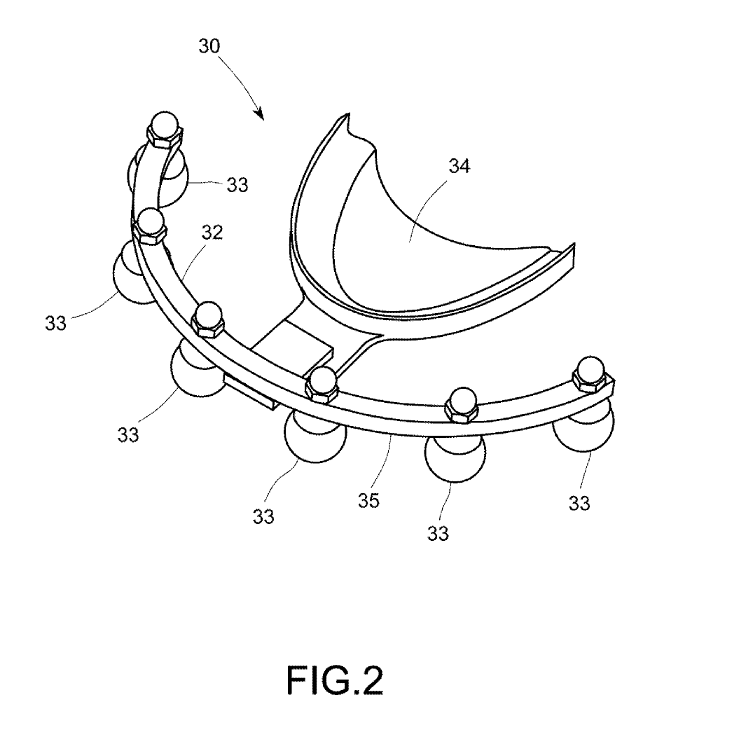

FIG. 2 shows a perspective view of a second embodiment of the invention, consisting of a device (30) for calibrating a quantitative computed tomography apparatus that includes a body (32) and six known-density elements (33), attached to the body (32) and made of materials and with densities not all equal to each other, and different from the body (32). The body (32) is shaped to be placed in a patient's mouth, with the known-density elements (33) arranged in the region of said person's teeth. In this embodiment, in particular, the body (32) has a mouth portion (34) shaped to be inserted into the person's mouth, preferably adapting to the internal shape of the mouth as shown in the figure, and an arched front portion (35) connected to the mouth portion (34) and intended to be arranged outside the mouth when the mouth portion (34) is inserted into a patient's mouth.

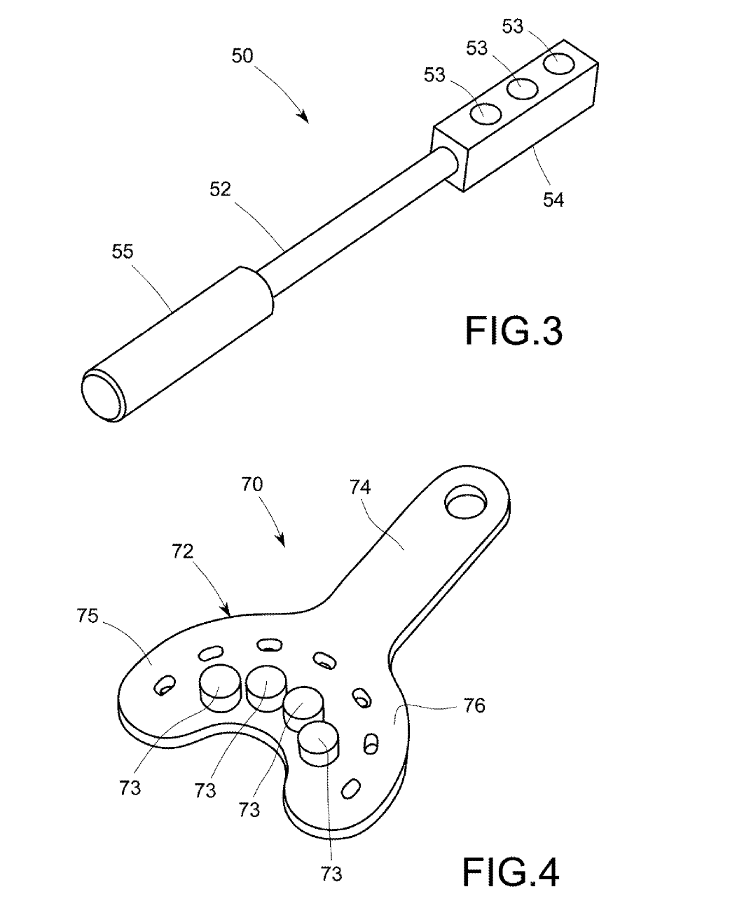

FIG. 3 shows a perspective view of a third embodiment of the invention, consisting in a device (50) for calibrating a quantitative computed tomography apparatus that includes a rod-shaped body (52) and three known-density elements (53) attached to the body (52). The three known-density elements (53) are made as inserts in a head (54) located at one end of the body (52). The body (52) is intended to be inserted into a patient's mouth to perform calibration of the quantitative computed tomography apparatus. These known-density elements (53) have different densities from the body (52) itself, and are made of different materials to the body (52), and all three preferably have different materials and densities from each other. Arranged at the opposite end of the body (52) is a handle (55) intended to protrude from the body (52) and allow a person—preferably the patient—to hold the body (52) by the handle (55) while inserting the head (54) inside the patient's mouth.

FIG. 4 shows a perspective view of a fourth embodiment of the invention, consisting in a device (70) for calibrating a quantitative computed tomography apparatus that includes a body (72) and four known-density elements (73), attached to the body (72), which are made of materials and with densities different from each other, and different from the body (72). The body (72) is shaped to be placed partially in the patient's mouth, the known-density elements (73) being inserted inside the patient's mouth and arranged in the region of the patient's teeth.

In this embodiment, the body (72) has an elongated portion (74) in the shape of a flat slat, and an end portion (75) arranged at one end of the elongated portion (74) and wider than the elongated portion (74). The known-density elements (73) are made as inserts in different material from the body (72), and protrude from the end portion (75) of the body (72), leaving a free surface (76) of the end portion (75) around the known-density elements (73). The free surface (76) is wide enough to be able to be bitten. Therefore, when the end portion (75) is inserted into the patient's mouth, the free surface (76) can be bitten and the known-density elements (73) firmly fixed in position in relation to the teeth, enabling quantitative computed tomography to be performed correctly.

As shown in the figure, the end portion (75) is preferably C-shaped so as to adapt to the internal contour of the person's teeth. The device (70) includes three known-density elements (73)—there can be more in alternative embodiments—also arranged to form a “C” similar to the shape of the end portion (75). This allows both the end portion (75) and the known-density elements (73) to have a shape and layout similar to the teeth and therefore the known-density elements (73) can be placed close to the patient's teeth.

The body (12, 32, 52, 72) of the embodiments described above is preferably made of polyacetal (POM-C), which is a plastic characterised by its hardness, stiffness and strength.

At the same time, at least one known-density element (13, 33, 53, 73) is made of polypropylene, ertacetal, PVDF or polytetrafluoroethylene (PTFE), which are plastic materials of different density and stiffness.

Preferably, the device (10, 30, 70) includes at least three known-density elements (13; 33; 73) made of different materials and densities, in which each material is either polypropylene, ertacetal, PVDF or PTFE. For example, the device (50) of FIG. 3 has exactly three known-density elements (53). By way of example, these known-density elements (53) can be made, for instance, of polypropylene, ertacetal and PVDF respectively.

The device (10, 30, 70) preferably includes at least four known-density elements (13, 33, 73), with at least one known-density element (13, 33, 73) made of polypropylene, at least another known-density element (13, 33, 73) made of ertacetal, at least another known-density element (13, 33, 73) made of PVDF and at least another known-density element (13, 33, 73) made of PTFE. These materials are interesting because they do not create artifacts in the radiographic examination and they can be sterilized.

The device (70) in the fourth embodiment, illustrated in FIG. 4, for example, includes four known-density elements (73), made respectively of polypropylene, ertacetal, PVDF and PTFE.

The known-density elements (13, 33, 53, 73) of the aforementioned embodiments preferably have the following densities: those made of polypropylene, a density of between 0.80 and 1.00 g/cm³; those made of ertacetal, a density of between 1.30 and 1.50 g/cm³; those made of PVDF, a density of between 1.60 and 1.90 g/cm³; those made of PTFE, a density of between 2.00 and 2.40 g/cm³ These density ranges enable an optimal conversion of the Hounsfield values of tomographic images into equivalent bone mineral density values in the spectrum of densities corresponding to bone tissue.

An example of the use of a device according to the invention for calibrating a quantitative computed tomography apparatus is explained in detail below. More specifically, an example of the use of the device (70) of FIG. 4 is explained.

Firstly, the person is placed in the quantitative computed tomography apparatus, suitably positioned to perform scanning. The person should preferably not have metal amalgams and implants, since calibration might otherwise be affected by them. Next, the device is held by the elongated portion (74) and the end portion (75) is inserted into the person's mouth. It is important to ensure that the person bites on the free surface (76) of the end portion (75), leaving the known-density elements (73) or cylinders in the tongue/palate area, i.e., in the area behind the teeth. Scanning of the person's mouth is then performed. After use, the device (70) is cleaned with a damp cloth and sterilized at a maximum of 121° C., after which it is ready to be used again. In the software application for managing and controlling the computed tomography apparatus, and for image processing and presentation, the study generated by scanning is opened. Either manually or automatically, the known-density elements (73) in the images are identified and, their density being known, the programme readjusts its calculations from Hounsfield (radiodensity) units to bone mineral density units (e.g. g/cm³) so that the bone mineral density results in the areas of the known-density elements (73) match the previously known densities of these known-density elements (73). This will cause readjustment of the grey levels of the entire image delivered by the software application, and will generate bone mineral density values of the scanned person's bones with optimum accuracy.

1. Device (10; 30; 50; 70) for the calibration of a quantitative computed tomography apparatus, characterized in that it includes:
a body (12; 32; 52; 72);
at least two known-density elements (13; 33; 53; 73) attached to the body (12; 32; 52; 72) and made of different materials and in different densities from each other and different from the body (12; 32; 52; 72); in which
the body (12; 32; 52; 72) is configured to be placed in the mouth or on another part of a person's head, with the known-density elements (13; 33; 53; 73) arranged in the region of said person's teeth.

2. Device (10), according to claim 1, characterised in that the body (12) has a cranial engagement portion (14) to support the device in the skull area of the person's head.

3. Device (30), according to claim 1, characterized in that the body (32) has a mouth portion (34) configured to be inserted into the person's mouth, and an arched front portion (35) attached to the mouth portion (34) and configured to be arranged outside the mouth when the mouth portion (34) is inserted into a patient's mouth, wherein the known-density elements (33) are fixed to said arched front portion (35).

4. Device (50), according to claim 1, characterized in that the body (52) is in the shape of a rod or bar, with the known-density elements (53) arranged at one end of the body (52).

5. Device (50), according to claim 4, characterized in that the body (52) comprises a handle (55) located at one end of the body (52) opposite the end where the known-density elements (53) are arranged.

6. Device (70), according to claim 1, characterized in that the body (72) has an elongated portion (74) in the shape of a flat slat and an end portion (75) arranged at one end of the elongated portion (74) and wider that the elongated portion (74), where the known-density elements (73) protrude from said end portion (75), a free surface (76) of this end portion (75) being delimited around the known-density elements (73), said free surface (76) being wide enough for it to be bitten.

7. Device (70), according to claim 6, characterized in that the end portion (75) is C-shaped so as to adapt to the internal contour of the person's teeth, and in that the device (70) comprises at least three known-density elements (73) also arranged to form a “C”, which is similar to the shape of the end portion (75).

8. Device (10; 30; 50; 70), according to claim 1, characterized in that the body (12; 32; 52; 72) is made of POM-C.

9. Device (10; 30; 50; 70), according to claim 1, characterized in that at least one known-density element (13; 33; 53; 73) is made of polypropylene.

10. Device (10; 30; 50; 70), according to claim 1, characterized in that at least one known-density element (13; 33; 53; 73) is made of ertacetal.

11. Device (10; 30; 50; 70), according to claim 1, characterized in that at least one known-density element (13; 33; 53; 73) is made of PVDF.

12. Device (10; 30; 50; 70), according to claim 1, characterized in that at least one known-density element (13; 33; 53; 73) is made of PTFE.

13. Device (10; 30; 50; 70), according to claim 1, characterized in that it comprises at least three known-density elements (13; 33; 53; 73) made of different materials and densities, wherein each material is one of polypropylene, ertacetal, PVDF and PTFE.

14. Device (10, 30, 70), according to claim 1, characterized in that it comprises at least four known-density elements (13, 33, 73), at least one known-density element (13, 33, 73) being made of polypropylene, at least another known-density element (13, 33, 73) being made of ertacetal, at least another known-density element (13, 33, 73) being made of PVDF and at least another known-density element (13, 33, 73) being made of PTFE.

15. Device (70), according to claim 1, characterized in that it comprises four known-density elements (73), made respectively of polypropylene, ertacetal, PVDF and PTFE.
